(19) United States
(12) Patent Application Publication
(10) Pub. No.: US 2008/0095870 A1
(43) Pub. Date: Apr. 24, 2008

(54) COMPOSITION OF MEDICINE, HEALTH-CARE PRODUCT AND COSMETICS FOR TREATING CHLOASMA

(76) Inventors: BOFANG CHAO, Changzhou (CN); Shuhua Gu, Changzhou (CN)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/865,683

(22) Filed: Oct. 1, 2007

(30) Foreign Application Priority Data
Oct. 21, 2006 (CN) 200610117403.X

(51) Int. Cl. A61K 36/756; A61K 36/00; A61P 17/00; A61K 36/284

(52) U.S. Cl. 424/769; 424/725; 424/773

(57) ABSTRACT

This invention relates to the composition of medicine, also a health-care product, cosmetics for treating chloasma and the method of the composition preparation and application. The composition contains active extracts of atractylodes lancea, semen coicis, and phellodendron amurense rupr, which are ruptured by solvent in weight ratio of 1:0.5-5:0.5-5.

### CROSS-REFERENCE TO RELATED APPLICATION

This application claims priority to Chinese patent application No. 200610117403.X, filed on Oct. 21, 2006, the teaching of which is incorporated herein in its entirety by reference.

### BACKGROUND OF THE INVENTION

1. Field of the Invention

This invention relates to a composition of medicine for treating skin disease, in a more accurate term, chloasma. It is a kind of health-care products, cosmetics, and it also includes a method of the composition preparation and its applications.

2. Description of the Background

Chloasma is a skin disease, which can be found on face caused by pigmentation; this skin disease can take place in both male and female, more in female. The chloasma also is named as “liver speckle”; it was described as “Mian Chen” in tradition Chinese. It looks like some brown spots or freckles that can be seen over the cheeks, forehead, zygomatic, nose and lips. It is butterfly shaped, the edge is clear, the surface is smooth, and there is no squama. It might be due to estrogen and progestin caused skin pigmentation, happened more in females. It also maybe caused by chronic wasting disease, such as tuberculosis, cancer, liver disease, and chronic alcoholism. It might be occurred in long-term taking drugs such as chlorpromazine, phenyloin sodium, and contraceptives and so on. Since it has no subjective symptoms, patients often delay to visit clinic. Major complains of outpatient mostly are chloasma affecting beauty appearances or with complications.

The theories of traditional Chinese medicine for treating this disease are explained here, which human body is considered as an integer, that face, five sensory organs and hairs are all each parts of the body. Fine facial appearance is only when the body is in healthy condition, Qi and blood are circulated, Yin and Yang of viscera are balanced, not depend on whitewash, the latter is considered as ‘pay attention on trifles and neglect essentials’. It is very well documented in ‘SHENG JI ZONG LU’, a traditional Chinese medicine record: “Qi and blood are the spirit of man, and heart is root of the blood; if the spirit changes, it can be seen from the face and if it is sufficient, it can be found from the bloods. So for therapeutic principle, at first we need nourish the Qi and blood, if you don't know this, only pay attention on whitewash, then you are far away from the truth.” The therapeutic principle of the case reported in the invention is holistic treatment, which is adjusting the circulating Qi and blood of the viscera, balancing the Yin and Yang. It is said in traditional Chinese medicine “the external is reflected by the internal status”, this disease is caused by dropout of the Qi and blood of the viscera, deficiency of the spleen and obstruction of damp-heat, so it appeared in face as Chloasma (the yellow is pertaining to “spleen”), if the spleen is ill, then it can not generating and sublimating, the origin of the blood is obstructed and will result in lacking control of Qi and blood; women are considering “blood” based, so the disease is appeared more in woman than man.

Chloasma is seriously affecting people's appearances; it gives great psychological pressure and psychic pain for patients. Currently there is no effective medicine, health-care products or cosmetics that resolving the problem.

### SUMMARY OF THE INVENTION

This invention relates to the composition of medicine, also a health-care product, cosmetics for treating chloasma and the method of the composition preparation and application. The composition contains active extracts of atractylodes lancea, semen coicis, and phellodendron amurense rupr, which are ruptured by solvent in weight ratio of 1:0.5-5:0.5-5.

### DETAILED DESCRIPTION OF THE INVENTION

One object of this invention is to provide medicinal extract that can effectively treating the chloasma.

A second object of the invention is to provide a composition medicine, a health-care product, cosmetics, which treating the chloasma, it contains medicinal active extract and the officinal carrier.

A third object of this invention is to provide the application of the medicinal active extract to prepare the medicine, health-care product and cosmetics for treating chloasma.

In traditional Chinese medicine (TCM), the atractylodes lancea is working as drying the damp and strengthen spleen, the semen coicis is working as dissipating dampness and strengthen spleen, the phellodendron amurense is working as QingHua (rupturing) damp-heat. Combine the three together can strengthening the spleen, drying the damp and clearing away the heat, the inventor of this invention found that the extract of the mixture have a favorable effect in treating the chloasma.

Therefore, this invention provided an active extract of the prescription of the atractylodes lancea, semen coicis and phellodendron amurense rupr. The weight ratio of atractylodes lancea, semen coicis and phellodendron amurense rupr is 1:0.5-5:0.5-5, optimal ratio is 1:0.5-2:0.5-2, the especially optimal ratio is 1:1:1.

The extraction solvent of this invention is water, organic solution and their mixture. The optimal selection of the extraction solvent is water, methyl alcohol, ethanol, acetone, butanol, formic acid, acetic acid, propionic acid, methyl chloride, methylene chloride, chloroform, carbon tetrachloride, dichloroethane, trichloroethane or their mixture. The especially excellent choose of the extracted solvent is water, the mixture of ethanol and water, the volume ratios of the water and ethanol is 1:0.1-10, the optimal ratio is 1:0.3-3. The especially optimal selection of the extraction solvent is water.

One optimal selection example of this invention, is that mixing the traditional Chinese medicinal raw materials, atractylodes lancea, semen coicis, and phellodendron amurense rupr in ratio of 1:1:1, extract three times by water or the add mixture of the water and the ethanol, then incorporate into the extraction solvent, concentrate and dry it, we can get the powder state of the active extract.

This invention also provided one composition medicine, a health-care product and cosmetics treating the chloasma, it contains: in term of the weight of the composition, 1-99% of the active extract and officinal carrier mentioned above. The composition medicine, a health-care product, cosmetics of this invention can be prepared in all different kind of dosage form for clinic application. Therefore, in the other hand this invention provided a preparation of medicine, a health-care product and cosmetics for effectively treating chloasma, wherein includes the effective dose of the active extract and the officinal carrier.

The dosage form of the medicine, the health-care product and cosmetics of this invention is oral or local application.

Oral application of this invention, the dosage form can be troche, liquor or suspension, powder dispersant or granules, latex, solid or soft capsule, syrup or elixir. Local application of this invention, the drug forms include liquor, suspension, lotion, cream, ointment, and gelatin.

The dosage form of oral application of this invention can be obtained by any known method for producing oral application in the composition of medicine, health-care product and cosmetics. This composition may contain one or more substances select from products below: sweetening agents, correctives, colorant and preservative, in order to provide pharmaceutically attractive appearance and dainty dosage form.

Troche contains active extract and nontoxic pharmaceutically acceptable excipient. These excipient are as following: inertia thinner, for example as calcium carbonate, sodium carbonate, lactose, calcium phosphate or sodium phosphate; granulate form and disintegrants are as following: microcrystalline cellulose, sodium carboxymethyl cellulose, cornstarch or algin; binders are as following: starch, glutin, polyvidone or acacia gum; lubes are as following: magnesium stearate, stearic acid or talcum powder.

The oral application dosage form of this invention also can be delivered as glutoid capsule, which mixed the active extract with the inertia solid thinner such as calcium carbonate, calcium phosphate and kaolin; or delivered as soft glutin capsule, which mixed the active extract with the water-soluble carriers such as polyethylene glycol or oil medium such as peanut oil, liquid paraffin or olive oil.

It contains active extract and the mixtures of solvent, excipient or dispersant for producing the solution, suspension, lotion, cream, ointment or gelation in the invention. The solvent includes water, alcohol and their mixture. The excipient include suspension, such as sodium carboxymethyl cellulose, methyl cellulose, hydroxypropoxyl methylcellulose, sodium alginate, polyvidone, tragacanth gum and acacia gum, starch, starch modified water-absorbent resin, fiber modified water-absorbent resin. The dispersant is natural phospholipids such as lecithin, or condensable product of alkylene oxide and fatty acid, such as polyoxyethylene stearates, or the condensable product of alkylene oxide and long chain adipic alcohol such as hepta-decaetileno-oxyel cetanol, or the condensable product of alkylene oxide and the meta-ester which derived from the fat and hexitol, such as polyoxyethane sorbitan monooleate.

The powder dispersant and granule forms of this invention are suitable for adding water to make a water suspension preparation before utilization. It supplies active extract with one or more kind of preservatives and dispersant, suspension. The appropriate dispersant and suspension are mentioned above. Other excipients can be sweetening agents, correctives and colorant, which can be preserved by adding antioxidant such as ascorbic acid.

The syrups and elixir of this invention can be prepared by sweetening agents such as glycerin, propanediol, sorbitol and sucrose. The dosage forms also include the buffer, preservative, correctant, colorant and antioxidant.

For local delivery application in the invention, a routine can be used, which mixing the active extract and medicinal carrier for routinely making the cream, ointment, liquor, suspension or gelatin.

The present invention also provided the application using the medicine, a health-care product and cosmetics to treat chloasma.

Use the active extract prepared the medicine, health-care product and cosmetics in the invention can effectively treat chloasma. The inventor delivered the composition medicine, health-care product and cosmetics to 80 patients with diagnosis of chloasma, observed the effect of treatment. Among 80 patients the youngest is 18 years old, the oldest 62 years old, there are 6 cases from age 21 to 30 years old, there are 46 cases from age 31 to 40 years old, and there are 26 cases from age 41 to 50 years old. Medication period is one month, no any adverse reactions were observed during the medication.

1. Standard of Therapeutic Effect

Improved: the chloasma is faint in color, the complications mitigated.

Significantly improved: the chloasma faded away, the complications are mostly disappeared.

Cured: the chloasma is disappeared, the complications recovered.

Failed: there is no change.

2. Result of the Therapeutic Effective

The improved 27 cases (33.75%), Markedly improved 22 cases (27.5%), cured 30 cases (37.5%), failed 1 cases (1.25%), total effective power is 98.75%.

### EXAMPLES

### Example 1

Case 1: Ms Zhu, female, 40 years old, worker. She has been taken estrogen since 1986, found the chloasma on her face symmetrically with butterfly shape, gradually develop to the area around the nose, mouth and lip, with clear edge and smooth surface, the color is getting deeper. She has no objective symptoms, only when she is constipated the chloasma will become much darker. She visit clinic because of chloasma affecting beauty appearance. After taking the medicine in the invention, the chloasma is disappeared. Follow-up the patient for six months, the chloasma was not recrudesce.

Case 2: Ms Lu female, 45 years old, worker. First visit to clinic on 5 Oct. 1989, the patient visited doctor because of the chloasma, headache with vertigo, and nausea. She has found chloasma on forehead and cheek with butterfly shape for 5 to 6 years. After medication of the medicine in the invention, the chloasma has disappeared. Follow-up the patient for six months, the chloasma was not recrudesced.

### Example 2

Place a TMC raw material of atractylodes lancea, semen coicis, and phellodendron amurense rupr 300 g each in ratio of 1:1:1 into 2.4 liters water, extracted for three times, then combined the extracted liquor together, after decompression, concentration and vacuum dehydration, obtained 12 grams of powder form of active extract

### Example 3

Place a TMC raw material of atractylodes lancea, semen coicis, and phellodendron amurense rupr 300 g each in ratio of 1:1:1 into 2.4 liters of 50% (V/V) ethanol, extracted for three times, then combined the extracted liquor together, after decompression, concentration and vacuum dehydration, obtained 8 grams of powder form of active extract.

### Example 4

Place a TMC raw material of atractylodes lancea, semen coicis, and phellodendron amurense rupr 300 g each in ratio of 1:1:1 into 2.4 liters of 50% (V/V) ethanol, extracted for three times, then combined the extracted liquor together, after decompression, concentration and vacuum dehydration, obtained 5 grams of powder form of active extract.

### Example 5

The following method is used for preparation of troche using the active extract in the invention: (all content is the % weight)

| | |
|---|---|
| Active extract | 30% |
| Microcrystalline cellulose | 66% |
| PVP-k30 | 2% |
| Magnesium stearate | 2% |

Mixed and uniformed the active extract, microcrystalline cellulose and PVP-k30, add ethanol, filtered in 12# sifter to get grannula, after through flow drying under 70° C., add the magnesium stearate, mixed to uniform to get the troche.

### Example 6

The following method is used for preparation of capsule form with the active extract of this invention (all the content is in % weight)

| | |
|---|---|
| Active extract | 25% |
| Calcium carbonates | 35% |
| Starch | 40% |

Mix the calcium carbonate and starch and cribration, then mixed with the active extract which dispersed by ethanol with 120 mesh screen, oven dry at 60-70° C. for 2 hs, then put the uniformed power to an empty capsule, ie.

### Example 7

The following method is used for preparation of guttate pills with the active extract of this invention: (all the content is in % weight)

| | |
|---|---|
| Active extract | 40% |
| Polyethylene glycol 600 | 60% |

Heat the polyethylene glycol 600 in a water bath until melted, added the active extract and mixed well to uniform, keep at 90° C. until utilization; preheat the devices of guttate pills, precooled the refrigerant of liquid paraffin to 10-15° C., drop at 50-70 drops per minute. Collected the guttate pills in the refrigerant, filtering the refrigerant, remove paraffin with filter paper, open-air dry till completion, ie.

### Example 8

Method for making oleamen is described as below:

Mixed 5 grams of the active extract and 95 grams of vaseline at the aseptic condition.

We claim:

1. An active extract, extracted using a solvent from the atractylodes lancea, semen coicis, and phellodendron amurense rupr with weight ratios of 1:0.5-5:0.5-5.

2. The active extract of claim 1, wherein the solvent is water, methyl alcohol, ethanol, acetone, butanol, formic acid, acetic acid, propionic acid, methyl chloride, methylene chloride, chloroform, carbon tetrachloride, dichloroethane, trichloroethane and their mixture.

3. The active extract of claim 1, wherein the weight ratios of atractylodes lancea, semen coicis, and phellodendron amurense rupr is 1:0.5-2:0.5-2, and the extracted solvent is water, ethanol or their mixture.

4. The active extract of claim 1, wherein the weight ratios of atractylodes lancea, semen coicis, and phellodendron amurense rupr is 1:1:1.

5. The active extract of claim 4, wherein the extracted solvent is water.

6. The active extract of claim 4, wherein the extracted solvent is the mixture of water and ethanol, and the mixing volume ratios of water and ethanol is 1:0.1-10.

7. The active extract of claim 6, wherein the volume ratio of water and ethanol is 1:0.3-30, the extracted solvent especially select water.

8. A composition of medicine, a health-care product and cosmetics, comprising the active extract according to claim 1 in an effective quantity for treating chloasma and an officinal carrier.

9. A composition of medicine, a health-care product and cosmetics, comprising the active extract according to claim 2 in an effective quantity for treating chloasma and an officinal carrier.

10. A composition of medicine, a health-care product and cosmetics, comprising the active extract according to claim 3 in an effective quantity for treating chloasma and an officinal carrier.

11. A composition of medicine, a health-care product and cosmetics, comprising the active extract according to claim 4 in an effective quantity for treating chloasma and an officinal carrier.

12. A composition of medicine, a health-care product and cosmetics, comprising the active extract according to claim 5 in an effective quantity for treating chloasma and an officinal carrier.

13. A composition of medicine, a health-care product and cosmetics, comprising the active extract according to claim 6 in an effective quantity for treating chloasma and an officinal carrier.

14. A composition of medicine, a health-care product and cosmetics, comprising the active extract according to claim 7 in an effective quantity for treating chloasma and an officinal carrier.

15. The composition of claim 8, wherein the composition of medicine, a health-care product, cosmetics is oral delivery dosage form, selected from troche, water solution or suspension, dispersible powder or granules, latex, solid or soft capsule, syrup or elixir.

16. The composition of claim 15, wherein the oral delivery drug form is troche, solid or soft capsule.

17. The composition of claim 8, wherein the local delivery dosage form can be selected from solution, suspension, lotion, cream, ointment or gel.

18. The composition of claim 8, wherein the transdermal delivery dosage form can be selected from solution, suspension, lotion, cream, ointment or gel.

19. The composition of claim 17, wherein the local delivery dosage form can be selected from solution, cream, ointment or gel.

20. A method of treating chloasma, comprising applying a composition comprising the active extract of claim 1 in an effective quantity to a patient.

21. A method of treating chloasma, comprising applying a composition comprising the active extract of claim 2 in an effective quantity to a patient.

22. A method of treating chloasma, comprising applying a composition comprising the active extract of claim 3 in an effective quantity to a patient.

23. A method of treating chloasma, comprising applying a composition comprising the active extract of claim 4 in an effective quantity to a patient.

24. A method of treating chloasma, comprising applying a composition comprising the active extract of claim 5 in an effective quantity to a patient.

25. A method of treating chloasma, comprising applying a composition comprising the active extract of claim 6 in an effective quantity to a patient.
